(19) United States
(12) Patent Application Publication
(10) Pub. No.: US 2023/0056953 A1
(43) Pub. Date: Feb. 23, 2023

(54) SURGICAL INSTRUMENTS HAVING A ROTATABLE BLADE MEMBER FOR TREATING TISSUE

(71) Applicant: Covidien LP, Mansfield, MA (US)

(72) Inventors: James R. Fagan, Erie, CO (US); Thomas E. Drochner, Longmont, CO (US); Michael B. Lyons, Boulder, CO (US); David J. Van Tol, Boulder, CO (US); Matthew S. Cowley, Frederick, CO (US)

(21) Appl. No.: 17/792,506

(22) PCT Filed: Jan. 7, 2021
(86) PCT No.: PCT/US2021/012486

§ 371 Date: Jul. 13, 2022

Related U.S. Application Data
(60) Provisional application No. 62/967,717, filed on Jan. 30, 2020.

(51) Int. Cl. A61B 17/295; A61B 17/32; A61B 17/29

(52) U.S. Cl. CPC A61B 17/295; A61B 17/2909; A61B 17/320092; A61B 2017/2905; A61B 2017/320075; A61B 2017/320094; A61B 2017/320095

(57) ABSTRACT

A surgical instrument for treating tissue includes an articulating elongated shaft (14), a drive shaft (28) extending through the elongated shaft and configured to rotate about a longitudinal axis defined by the drive shaft, and an end effector assembly (100) coupled to a distal end portion of the elongated shaft. The end effector assembly includes a jaw member (110) and a blade member (112) configured to rotate in response to a rotation of the drive shaft to treat tissue disposed between the jaw member and the blade member.

### FIELD

The present technology is generally related to surgical instruments and, more particularly, to tissue treating mechanisms for use with surgical end effector assemblies, instruments, and systems.

### BACKGROUND

A surgical forceps is a pliers-like instrument that relies on mechanical action between jaw members of its end effector assembly to grasp, clamp, and constrict tissue. Some surgical forceps utilize both mechanical clamping action and energy to accurately sever the tissue. Accordingly, many surgical forceps incorporate a knife, an ultrasonic blade, or other suitable cutting members utilized to effectively treat the tissue clamped by the end effector assembly.

During laparoscopic or endoscopic surgical procedures, access to a surgical site is achieved through a small incision or through a narrow cannula inserted through a small entrance wound in a patient. Because of limited area available to access the surgical site, many surgical forceps include mechanisms for articulating the end effector assembly thereof in relation to a body portion of the forceps to improve access to tissue to be treated.

### SUMMARY

The techniques of this disclosure generally relate to surgical instruments for sealing and/or cutting tissue. In accordance with aspects of the disclosure, the surgical instrument includes an elongated shaft, a drive shaft extending through the elongated shaft, and an end effector assembly coupled to a distal end portion of the elongated shaft. The distal end portion of the elongated shaft is configured to articulate relative to a proximal end portion of the elongated shaft, and the drive shaft is configured to rotate about a longitudinal axis defined by the drive shaft. The drive shaft has a proximal end portion configured to be operably coupled to a drive motor. The end effector assembly includes a jaw member pivotable relative to the distal end portion of the elongated shaft, and a blade member opposing the jaw member. The blade member is non-rotationally supported on a distal end portion of the drive shaft, such that the blade member is configured to rotate in response to a rotation of the drive shaft to treat tissue disposed between the jaw member and the blade member via friction created by the rotational motion of the drive shaft.

In aspects, the blade member may have a cylindrical configuration.

In aspects, the drive shaft may be flexible along a length thereof, such that the drive shaft flexes as the distal end portion of the elongated shaft articulates.

In aspects, the distal end portion of the elongated shaft may be configured to articulate from a first position, in which the end effector assembly is parallel with a longitudinal axis defined by the elongated shaft, and at least one second position, in which the end effector assembly is offset from the longitudinal axis defined by the elongated shaft.

In aspects, the drive shaft may have an intermediate portion interconnecting the proximal and distal end portions of the drive shaft. The proximal and distal end portions of the drive shaft may be rigid and the intermediate portion being flexible.

In aspects, the distal end portion of the elongated shaft may have an articulating section. The intermediate portion of the drive shaft may be disposed within the articulating section.

In aspects, the distal end portion of the elongated shaft may have a rigid distal section extending distally from the articulating section. The proximal end portion of the elongated shaft may be rigid and extend proximally from the articulating section.

In aspects, the proximal end portion of the drive shaft may be received in the proximal end portion of the elongated shaft. The distal end portion of the drive shaft may be received in the rigid distal section.

In aspects, the drive shaft may have a universal joint interconnecting the proximal and distal end portions of the drive shaft.

In aspects, the proximal and distal end portions of the drive shaft may each be rigid along their length.

In aspects, the surgical instrument may further include a clutch mechanism detachably coupling the proximal end portion of the drive shaft and the drive motor.

In accordance with another aspect of the disclosure, a hand-held surgical instrument is provided and includes a handle assembly, an elongated shaft, a drive shaft, and an end effector assembly. The handle assembly has a handle housing, a drive motor disposed within the handle housing, an articulation switch movably coupled to the handle housing, and a trigger movably coupled to the handle housing. The elongated shaft has a proximal end portion coupled to the handle housing, and a distal end portion configured to articulate relative to the proximal end portion in response to an actuation of the articulation switch. The drive shaft extends longitudinally along the elongated shaft and is configured to rotate about a longitudinal axis defined by the drive shaft. The drive shaft has a proximal end portion operably coupled to the drive motor. The end effector assembly is coupled to the distal end portion of the elongated shaft and includes a jaw member and a blade member opposing the jaw member. The jaw member is pivotable relative to the distal end portion of the elongated shaft. The blade member is coupled to a distal end portion of the drive shaft, such that the blade member is configured to rotate in response to an actuation of the trigger to treat tissue disposed between the jaw member and the blade member.

In accordance with yet another aspect of the disclosure, a surgical instrument for treating tissue is provided and includes an elongated shaft, a tubular shaft extending through the elongated shaft, and a blade member coupled to a distal end portion of the tubular shaft. The tubular shaft defines a lumen configured to receive a fluid, and the blade member defines an internal chamber in fluid communication with the lumen. The blade member has a plurality of vanes, such that the blade member is configured to rotate relative to the tubular shaft in response to the fluid moving over the plurality of vanes.

In aspects, the elongated shaft may have a proximal end portion, and a distal end portion configured to articulate relative to the proximal end portion.

In aspects, the tubular shaft may be flexible along a length thereof, such that the drive shaft flexes as the distal end portion of the elongated shaft articulates.

In aspects, the surgical instrument may further include a jaw member pivotable relative to the distal end portion of the elongated shaft and configured to clamp tissue between the jaw member and the blade member.

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements and:

FIG. 1A is a perspective view illustrating a hand-held surgical instrument including a jaw member in an opened position relative to a rotatable blade member;

FIG. 1B is a perspective view illustrating the surgical instrument of FIG. 1A in an articulation configuration with the jaw member in a closed position relative to the blade member;

FIG. 2 is a side view illustrating a drive assembly of the surgical instrument of FIGS. 1A and 1B for driving a rotation of the blade member;

FIG. 3 is a side view illustrating another embodiment of a drive assembly of the surgical instrument of FIGS. 1A and 1B for driving a rotation of the blade member;

FIG. 4 is a side view illustrating another embodiment of a drive assembly of the surgical instrument of FIGS. 1A and 1B for driving a rotation of the blade member;

FIG. 5 is a side view illustrating another embodiment of a drive assembly for use in the surgical instrument of FIGS. 1A and 1B; and

FIG. 6 is a schematic illustration of a robotic surgical system incorporating any of the disclosed drive assemblies.

### DETAILED DESCRIPTION

The disclosure is generally directed to a minimally invasive surgical instrument for grasping and treating tissue using a rotating blade member. Some surgical instruments have a blade member in communication with an ultrasonic transducer for vibrating the blade member at ultrasonic frequencies suitable for treating (e.g., cutting and/or sealing) tissue contacted by the blade member. To transfer ultrasonic energy to the blade member, a rigid waveguide typically interconnects the ultrasonic transducer and the blade member. Due to the rigid nature of most waveguides, it is difficult to incorporate an articulating function into ultrasonic surgical instruments. The surgical instrument of the disclosure cures these and other drawbacks of ultrasonic surgical instruments.

The surgical instrument of the disclosure may be hand-held or have components configured to be operated by a robotic system. The surgical instrument includes an elongated shaft having an end effector assembly coupled to a distal end portion thereof. The elongated shaft has a flexible or bendable articulating section that allows the distal end portion and the attached end effector assembly to articulate in a plurality of directions. The end effector assembly has a pivotable jaw member and a rotatable blade member in opposing relation with the jaw member. Tissue may be grasped between the jaw member and the blade member, whereby the blade member is rotated to treat the grasped tissue due to the friction generated between the rotating blade member and the grasped tissue. The blade member is driven by a rotatable drive shaft extending through the elongated shaft. The drive shaft has a flexible, pivotable, or bendable portion that articulates with an articulation of the elongated shaft while also allowing for the transfer of rotational forces through the elongated shaft and to the blade member.

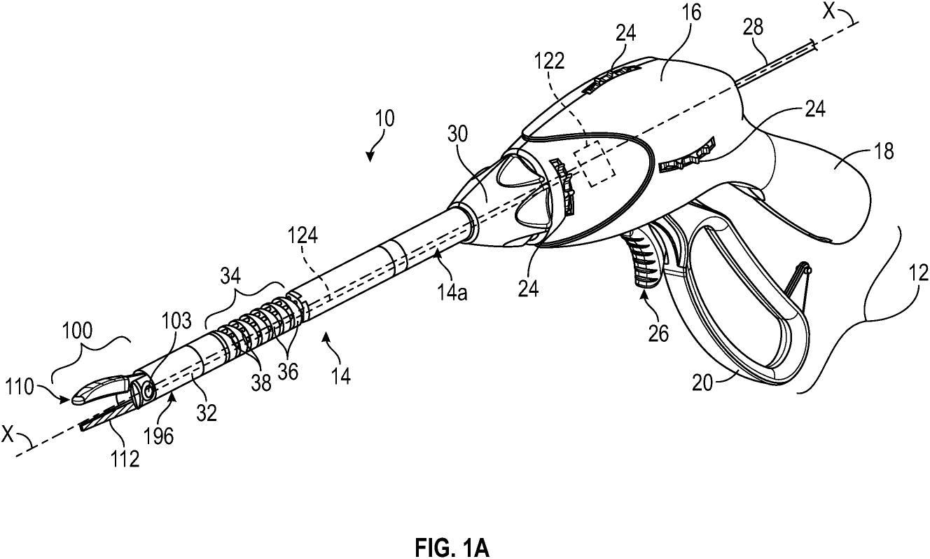

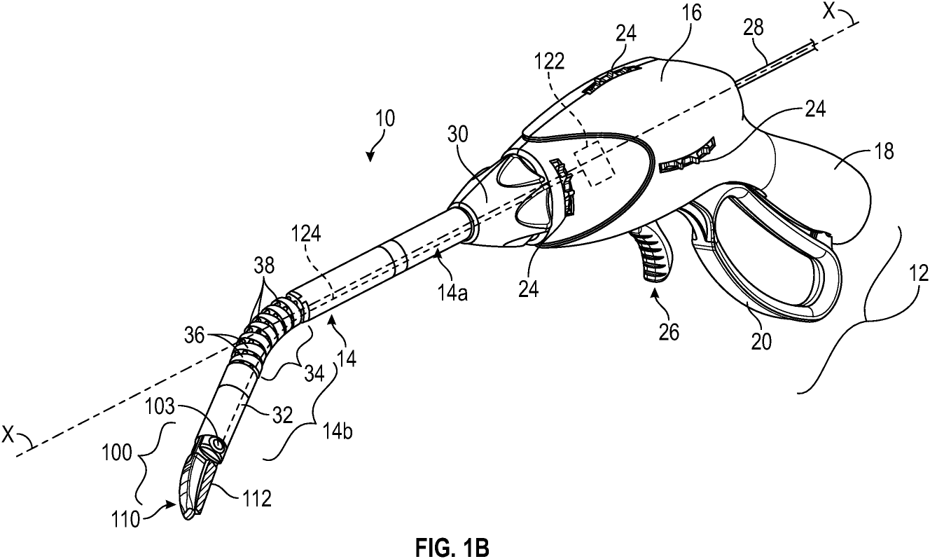

Referring generally to FIGS. 1A and 1B, an endoscopic, hand-held surgical instrument 10 includes a handle assembly 12, an endoscopic portion, such as, for example, an elongated shaft 14 extending distally from the handle assembly 12, and an end effector assembly 100 coupled to a distal end portion 14b of the elongated shaft 14. The handle assembly 12 includes a handle housing 16 having a fixed handle 18 integrally associated therewith, and a movable handle 20 movable relative to the fixed handle 18. The movable handle 20 is operably coupled to a drive assembly (not shown) configured to impart movement of a jaw member 110 of the end effector assembly 100 about a pivot 103 relative to a blade member 112 of the end effector assembly 100.

The jaw member 110 is configured to move between a spaced-apart position (FIG. 1A) and an approximated position (FIG. 1B) to grasp tissue between the jaw member 110 and the blade member 120. As shown in FIG. 1A, the movable handle 20 is disposed in a spaced-apart position relative to the fixed handle 18 and, correspondingly, the end effector assembly 100 is disposed in the spaced-apart position. The movable handle 20 is depressible from the spaced-apart position to a depressed position corresponding to the approximated position of the end effector assembly 100 (FIG. 1B). In aspects, the surgical instrument 10 may be devoid of the jaw member 110.

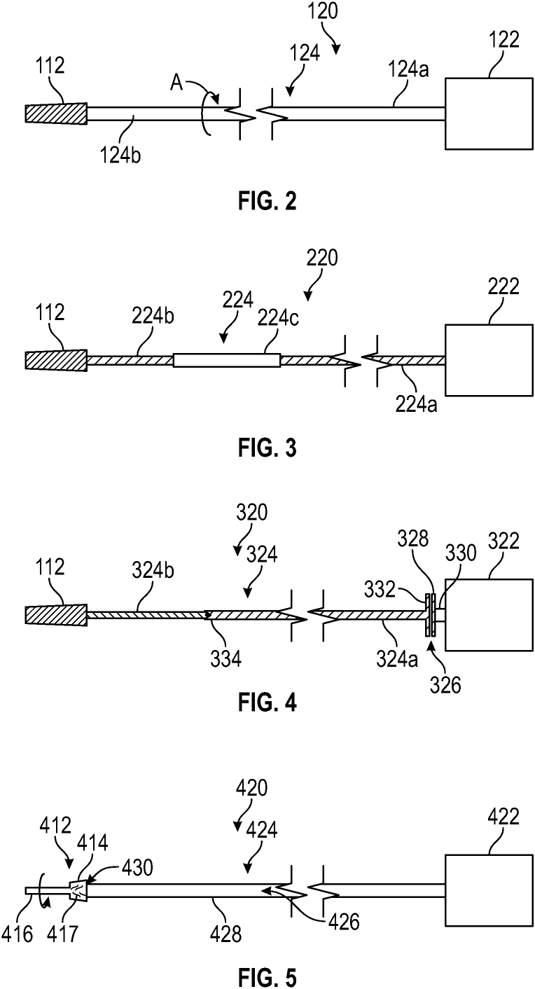

The handle assembly 12 further includes a plurality of articulation actuators 24 and a trigger 26 each movably coupled to the handle housing 16. The articulation actuators 24 are configured to effectuate an articulation of the end effector assembly 100 between a non-articulated position (FIG. 1A), in which the end effector assembly 100 is coaxial or otherwise parallel with the longitudinal axis “X” defined by the elongated shaft 14, and at least one articulated position (FIG. 1B), in which the end effector assembly 100 is offset from the longitudinal axis “X” defined by the elongated shaft 14. The trigger 26 is pivotable relative to the handle housing 16 and is configured to actuate a drive motor 122 (FIG. 2) to drive a rotation of the blade member 112 of the end effector assembly 100. In aspects, the drive motor 122 may be configured to increase the rotational speed of the blade member 112 as the trigger 26 approximates the fully-actuated position. In aspects, detents may be provided on the trigger 26 to provide a user with tactile feedback to indicate the rotational speed of the blade member 112. As an alternative to a pivoting trigger 26, a slide trigger, push-button, toggle switch, or other suitable actuator may be provided. The surgical instrument 10 may be powered via an internal battery (not explicitly shown) or, alternately, by an external power source via a cable 28 or a cable-free connection to a robot arm.

The proximal end portion 14a of the elongated shaft 14 is non-rotationally coupled to a rotatable knob housing 30, which is rotatably coupled to the handle housing 16, such that the elongated shaft 14 and the attached end effector assembly 100 are configured to rotate about a longitudinal axis “X” defined by the elongated shaft 14. The proximal end portion 14a of the elongated shaft 14 may be rigid along its length to maintain a linear configuration during use. The distal end portion 14b of the shaft 14 has a rigid distal section 32 connected to the end effector assembly 100 and an articulating section 34 disposed between the rigid distal section 32 and the proximal end portion 14a. The articulating section 34 includes a plurality of articulating links 36 having a plurality of articulation cables 38 extending therethrough. Each cable 38 is operably engaged at its distal end to the rigid distal section 32 and at its proximal end to one of the articulation actuators 24 so as to enable articulation of the rigid distal section 32 and, thus, the end effector assembly 100, relative to the proximal end portion 14a upon actuation of one or more of the articulation actuators 24. In some aspects, the articulating section 34 and the articulation actuators 24 may be omitted, such that the elongated shaft 14 does not articulate.

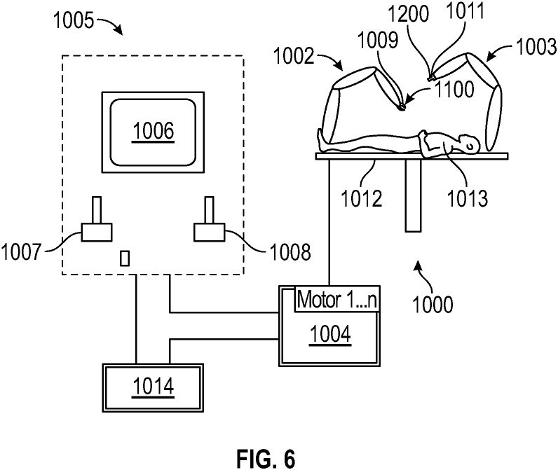

With reference to FIG. 2, the surgical instrument 10 includes a drive assembly 120 for driving a rotation of the blade member 112 of the end effector assembly 100. The drive assembly 120 includes a drive motor 122 received in the handle housing 16 (FIG. 1A) or in a robot arm 1002 (FIG. 6) and a drive shaft 124 extending through the elongated shaft 14 (FIG. 1A). The blade member 112 is integrally formed with or otherwise attached to a distal end portion 124b of the drive shaft 124 and is non-rotatable relative to the drive shaft 124. The blade member 112 may have a cylindrical configuration and be fabricated from a metal having a coarse outer surface (e.g., knurls, splines, abrasive material having a grit size from about 20 to about 1500, etc.) to increase the frictional engagement between the blade member 112 and tissue. In aspects, the blade member 112 may assume any suitable shape, such as, for example, a rectangle, a triangle, a sphere, or the like, and may be fabricated from any suitable material, such as, for example, plastics, metals, etc. The blade member 112 may have a smooth outer surface or surface projections, such as, for example, a plurality of spikes extending from the outer surface thereof. In aspects, spikes or other suitable surface features on the blade member 112 may be formed via any suitable process, such as chemically etching.

The drive motor 122 may be an electric motor operably coupled to a proximal end portion 124a of the drive shaft 124. The drive motor 122 drives a rotation of the drive shaft 124 about a longitudinal axis of the drive shaft 124. The proximal end portion 124a of the drive shaft 124 may be directly, operably coupled to the drive motor 122 or indirectly, operably coupled to the drive motor 122 via a series of gears, belts, screws, linkages, or the like. The drive shaft 124 is fabricated from a flexible material, such as, for example, rubber, plastics, metals, etc., to allow the drive shaft 124 to flex or otherwise bend during articulation of the articulating section 34 (FIG. 1B) of the elongated shaft 14 while also maintaining the ability to transfer rotational forces from the drive motor 122 to the blade member 112. In aspects, the drive shaft 124 may be a tube, a non-cannulated shaft, or include a bundle of metal wires.

In operation, tissue is positioned between the jaw member 110 and the blade member 112 with the end effector assembly 100 in the spaced-apart position, as shown in FIG. 1A. To treat or otherwise seal/cut the tissue during a surgical procedure, the movable handle 20 is actuated toward the depressed position (FIG. 1B), whereby the jaw member 110 is pivoted toward the blade member 112 to close the end effector assembly 100 about the tissue. With the end effector assembly 100 in the closed position, as shown in FIG. 1B, the trigger 26 may be actuated to activate the drive motor 122 of the drive assembly 120. The drive motor 122 drives a rotation of the drive shaft 124, in the direction indicated by arrow “A” in FIG. 2, thereby inducing a corresponding rotation of the blade member 112. The rotational speed of the blade member 112 may be sufficient to treat the tissue due to the high friction between the blade member 112 and the tissue. In aspects, the high friction between the blade member 112 and the tissue may be sufficient only to cut the tissue and RF energy or another suitable type of energy may be applied to the tissue to seal the tissue. In aspects, the rotational speed of the blade member 112 may be from about 1,000 revolutions per minute (“RPM”) to about 350,000 RPM, and in some aspects, from about 20,000 RPM to about 40,000 RPM. In some aspects, the surgical instrument 10 may cut through tissue without having to clamp the tissue between the jaw member 110 and the blade member 112. In aspects, the distal-facing tip or edge of the blade member 112 may be oriented perpendicular to the tissue surface and rotated to form an opening or hole in the tissue. It is contemplated that the distal-facing edge of the blade member 112 may have a coarse outer surface to enhance the frictional engagement between the distal-facing edge and the tissue surface.

FIG. 3 illustrates another embodiment of a drive assembly 220 for use in the surgical instrument 10 of FIGS. 1A and 1B instead of the drive assembly 120 of FIG. 2. The drive assembly 220 includes an electric drive motor 222 and an elongated drive shaft 224 operably coupled to the drive motor 222. The blade member 112 is integrally connected to or otherwise attached to a distal end portion 224b of the drive shaft 224. The drive shaft 224 of FIG. 3 differs from the drive shaft 124 of FIG. 2 by being made up of three discrete longitudinal segments, namely a proximal end portion 224a, a distal end portion 224b, and an intermediate portion 224c interconnecting the proximal and distal end portions 224a, 224b.

The proximal and distal end portions 224a, 224b are both rigid along their lengths (e.g., the proximal and distal end portions 224a, 224b are configured to resist twisting and bending), whereas the intermediate portion 224c is flexible along its length to allow for flexing of the intermediate portion 224c during articulation of the articulating section 34 (FIG. 1B). In particular, upon assembly of the drive shaft 224 into the elongated shaft 14 (FIG. 1B), the proximal end portion 224a of the drive shaft 224 extends through the rigid proximal end portion 14a of the elongated shaft 14, the intermediate portion 224c of the drive shaft 224 extends through the articulating section 34 of the elongated shaft 14, and the distal end portion 224b of the drive shaft 224 extends through the rigid distal section 32 of the distal end portion 14b of the elongated shaft 14.

FIG. 4 illustrates another drive assembly 320 suitable for use in the surgical instrument 10 of FIGS. 1A and 1B instead of the drive assembly 120 of FIG. 2. The drive assembly 320 includes an electric drive motor 322 and an elongated drive shaft 324 operably coupled to the drive motor 322 via a clutch mechanism 326. The blade member 112 is integrally connected to or otherwise attached to a distal end portion 324b of the drive shaft 324. The clutch mechanism 326 detachably couples the proximal end portion 324a of the drive shaft 324 to the drive motor 322. The clutch mechanism 326 may include a first plate 328 attached to a drive rod 330, which is rotationally driven by the drive motor 322, and a second plate 332 fixed to the proximal end portion 324a of the drive shaft 324. The first and second plates 328, 332 may each have opposing, high friction surfaces that when engaged with one another allow for the transfer of rotational forces therebetween.

The drive rod 330 may be axially movable relative to the drive motor 322 to selectively engage and disengage the first and second plates 328, 332. In other aspects, the drive shaft 324 may be axially movable to selectively engage and disengage the first and second plates 328, 332. It is contemplated that the clutch mechanism 326 may have various configurations, such as a centrifugal clutch, a hydraulic clutch, an electromagnetic clutch, a diaphragm clutch, etc. In aspects, the clutch mechanism 326 may be incorporated into any of the drive shafts described herein.

The drive shaft 324 may further include a universal joint 334 that interconnects the proximal and distal end portions 324a, 324b of the drive shaft 324. The universal joint 334 is received in the articulating section 34 (FIG. 1B) of the elongated shaft 14 to allow the distal end portion 324b of the drive shaft 324 to articulate with the distal end portion 14b of the elongated shaft 14. The proximal and distal end portions 324a, 324b of the drive shaft 324 may each be rigid along their lengths to resist twisting or bending of the proximal and distal end portions 324a, 324b of the drive shaft 324 during rotation of the drive shaft 324.

In operation of the drive assembly 320, prior to treating tissue and upon powering on the surgical instrument 10, the drive motor 322 may be automatically activated to rotate the drive rod 330 and the attached first plate 328 of the clutch mechanism 326 at a predetermined rate while the drive shaft 324 is disengaged from the drive motor 322. To treat or otherwise cut tissue, the clutch mechanism 326 is engaged by actuating the trigger 26, whereby the first and second plates 328, 332 are non-rotationally engaged. With the drive shaft 324 non-rotationally coupled to the drive rod 330, the rotational motion of the drive rod 330 is transferred to the blade member 112 via the drive shaft 324. In this way, when the treating function of the blade member 112 is desired, the drive shaft 324 and the attached blade member 112 may assume a rotational speed at a suitable rpm instantaneously upon engaging the already-rotating first plate 328 with the second plate 332 of the drive shaft 324.

FIG. 5 illustrates another assembly 420 suitable for use in the surgical instrument 10 of FIGS. 1A and 1B instead of the drive assembly 120 of FIG. 2. The drive assembly 420 may be configured similarly to a pneumatic turbine and includes a source of pressurized fluid 422 (e.g., a canister of compressed CO₂), a tubular shaft 424, such as, for example, a hose extending through the elongated shaft 14 (FIG. 1A), and a blade member 412. The tubular shaft 424 is flexible along its length to allow for articulation of the elongated shaft 14 and defines a lumen 426 therethrough in communication with the source of pressurized fluid 422. In aspects, the trigger 26 (FIG. 1A) of the handle assembly 12 may be operably coupled to a valve (not explicitly shown) of the source of pressurized fluid 422 to selectively release a compressed fluid from the source of pressurized fluid 422 into the lumen 426 of the tubular shaft 424. In aspects, the valve may be located remotely, such as in a robot arm (e.g., robot arm 1002 of FIG. 6).

The blade member 412 is rotatably coupled to a distal end portion 428 of the tubular shaft 424. The blade member 412 has a proximal body portion 414 and an elongated tissue-treating segment or shaft 416 extending from the proximal body portion 414. The proximal body portion 414 defines an internal chamber 430 in fluid communication with the lumen 426 of the tubular shaft 424. The proximal body portion 414 of the blade member 412 also defines a plurality of vanes 417 (e.g., fins or blades) circumferentially disposed about the blade member 412 and in fluid communication with the lumen 426 of the tubular shaft 424. The vanes 417 are arranged and structured such that the blade member 412 rotates about a longitudinal axis defined by the rigid distal section 32 (FIG. 1B) as fluid passes from within the lumen 426 and out of the blade member 412 via the vanes 417.

Referring generally to FIG. 6, a robotic surgical system exemplifying the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to the end effector assembly 100 (FIGS. 1A and 1B), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical instrument for treating tissue, comprising:
an elongated shaft having a proximal end portion and a distal end portion configured to articulate relative to the proximal end portion;
a drive shaft extending through the elongated shaft and configured to rotate about a longitudinal axis defined by the drive shaft, the drive shaft having a proximal end portion configured to be operably coupled to a drive motor; and
an end effector assembly coupled to the distal end portion of the elongated shaft and including:
a jaw member pivotable relative to the distal end portion of the elongated shaft; and
a blade member opposing the jaw member, wherein the blade member is non-rotationally supported on a distal end portion of the drive shaft, such that the blade member is configured to rotate in response to a rotation of the drive shaft to treat tissue disposed between the jaw member and the blade member.

2. The surgical instrument according to claim 1, wherein the blade member has a cylindrical configuration.

3. The surgical instrument according to claim 1, wherein the drive shaft is flexible along a length thereof, such that the drive shaft flexes as the distal end portion of the elongated shaft articulates.

4. The surgical instrument according to claim 3, wherein the distal end portion of the elongated shaft is configured to articulate from a first position, in which the end effector assembly is parallel with a longitudinal axis defined by the elongated shaft, and at least one second position, in which the end effector assembly is offset from the proximal end portion of the elongated shaft.

5. The surgical instrument according to claim 1, wherein the drive shaft has an intermediate portion interconnecting the proximal and distal end portions of the drive shaft, the proximal and distal end portions of the drive shaft being rigid and the intermediate portion being flexible.

6. The surgical instrument according to claim 5, wherein the distal end portion of the elongated shaft has an articulating section, the intermediate portion of the drive shaft being disposed within the articulating section.

7. The surgical instrument according to claim 6, wherein the distal end portion of the elongated shaft has a rigid distal section extending distally from the articulating section, and the proximal end portion of the elongated shaft is rigid and extends proximally from the articulating section.

8. The surgical instrument according to claim 7, wherein the proximal end portion of the drive shaft is received in the proximal end portion of the elongated shaft and the distal end portion of the drive shaft is received in the rigid distal section of the elongated shaft.

9. The surgical instrument according to claim 1, wherein the drive shaft has a universal joint interconnecting the proximal and distal end portions of the drive shaft.

10. The surgical instrument according to claim 9, wherein the proximal and distal end portions of the drive shaft are each rigid along their length.

11. The surgical instrument according to claim 1, further comprising a clutch mechanism detachably coupling the proximal end portion of the drive shaft and the drive motor.

12. A hand-held surgical instrument, comprising:
a handle assembly including:
a handle housing;
a drive motor disposed within the handle housing;
an articulation switch movably coupled to the handle housing; and
a trigger movably coupled to the handle housing;
an elongated shaft having a proximal end portion coupled to the handle housing, and a distal end portion configured to articulate relative to the proximal end portion in response to an actuation of the articulation switch;
a drive shaft extending longitudinally along the elongated shaft and configured to rotate about a longitudinal axis defined by the drive shaft, the drive shaft having a proximal end portion operably coupled to the drive motor; and
an end effector assembly coupled to the distal end portion of the elongated shaft and including:
a jaw member pivotable relative to the distal end portion of the elongated shaft; and
a blade member opposing the jaw member, wherein the blade member is coupled to a distal end portion of the drive shaft, such that the blade member is configured to rotate in response to an actuation of the trigger to treat tissue disposed between the jaw member and the blade member.

13. The hand-held surgical instrument according to claim 12, wherein the drive shaft is flexible along a length thereof, such that the drive shaft flexes as the distal end portion of the elongated shaft articulates.

14. The hand-held surgical instrument according to claim 12, wherein the distal end portion of the elongated shaft is configured to articulate from a first position, in which the end effector assembly is parallel with a longitudinal axis defined by the elongated shaft, and at least one second position, in which the end effector assembly is offset from the longitudinal axis defined by the elongated shaft.

15. The hand-held surgical instrument according to claim 12, wherein the drive shaft has an intermediate portion interconnecting the proximal and distal end portions of the drive shaft, and the proximal and distal end portions of the drive shaft are rigid and the intermediate portion is flexible.

16. The hand-held surgical instrument according to claim 15, wherein the distal end portion of the elongated shaft has an articulating section and the intermediate portion of the drive shaft is disposed within the articulating section.

17. The hand-held surgical instrument according to claim 16, wherein the distal end portion of the elongated shaft has a rigid distal section extending distally from the articulating section, and the proximal end portion of the elongated shaft is rigid and extends proximally from the articulating section.

18. The hand-held surgical instrument according to claim 17, wherein the proximal end portion of the drive shaft is received in the proximal end portion of the elongated shaft, and the distal end portion of the drive shaft is received in the rigid distal section of the elongated shaft.

19. The hand-held surgical instrument according to claim 12, wherein the drive shaft has a universal joint interconnecting the proximal and distal end portions of the drive shaft.

20. The hand-held surgical instrument according to claim 19, wherein the proximal and distal end portions of the drive shaft are each rigid along their length.

21. The hand-held surgical instrument according to claim 12, further comprising a clutch mechanism detachably coupling the proximal end portion of the drive shaft and the drive motor.

22. A surgical instrument for treating tissue, comprising:
an elongated shaft;
a tubular shaft extending through the elongated shaft and defining a lumen configured to receive a fluid; and
a blade member coupled to a distal end portion of the tubular shaft and defining an internal chamber in fluid communication with the lumen, wherein the blade member has a plurality of vanes, such that the blade member is configured to rotate relative to the tubular shaft in response to the fluid moving over the plurality of vanes.

23. The surgical instrument according to claim 22, wherein the elongated shaft has a proximal end portion and a distal end portion configured to articulate relative to the proximal end portion.

24. The surgical instrument according to claim 23, wherein the tubular shaft is flexible along a length thereof, such that the drive shaft flexes as the distal end portion of the elongated shaft articulates.

25. The surgical instrument according to claim 22, further comprising a jaw member pivotable relative to the distal end portion of the elongated shaft and configured to clamp tissue between the jaw member and the blade member.
